(19) United States
(12) Patent Application Publication
(10) Pub. No.: US 2013/0309478 A1
(43) Pub. Date: Nov. 21, 2013

(54) PIECE OF CLOTHING

(71) Applicant: Fraunhofer-Gesellschaft zur Foerderung der angewandten Forschung e.V., Munich (DE)

(72) Inventors: Sabine TRUPP, Saalfeld (DE); Matthias STICH, Regensburg (DE); Gerhard MOHR, Regensburg (DE)

(73) Assignee: Fraunhofer-Gesellschaft zur Foerderung der angewandten Forschung e.V., Munich (DE)

(21) Appl. No.: 13/955,306

(22) Filed: Jul. 31, 2013

Related U.S. Application Data
(63) Continuation of application No. PCT/EP2012/051620, filed on Feb. 1, 2012.

(30) Foreign Application Priority Data
Feb. 2, 2011 (DE) 102011003517.6

(51) Int. Cl. G01N 33/18; A41D 1/00

(52) U.S. Cl. CPC G01N 33/18; A41D 1/00
USPC 428/221

(57) ABSTRACT

An easy way for a person to utilize the indicating character of body perspiration for recognizing possible diseases is realized by using a piece of clothing as a carrier for a corresponding indicator dye, without additional stress or strain in everyday life or additional effort of a general nature, by bonding the indicator dye to the fiber of the piece of clothing by means of a chemical bond such as an atomic bond or a covalent bond.

### CROSS-REFERENCE TO RELATED APPLICATIONS

This application is a continuation of copending International Application No. PCT/EP2012/051620, filed Feb. 1, 2012, which is incorporated herein by reference in its entirety, and additionally claims priority from German Application No. DE 102011003517.6-43, filed Feb. 2, 2011, which is also incorporated herein by reference in its entirety.

### BACKGROUND OF THE INVENTION

The present invention relates to pieces of clothing and, on the other hand, to the detection of analytes, like, e.g., to pH value detection.

With sports activities or other kinds of effort you easily start to sweat. Often, people are not aware of the fact that they lose a considerable amount of water through sweating as the evaporation of sweat results in a cooling effect. If too much water is lost, this may lead to dizziness, cardiac arrhythmia, kidney failure or unconsciousness, however. No method is known which gives direct information to the (athletically) active person on the water loss which has occurred.

Further, in people's sweat a variety of analytes can be detected which may, on the one hand, give information on fitness and even on existing diseases. Current methods for detecting analytes in body perspiration are being developed and necessitate the extensive use of machines and do not enable any direct on-site diagnostics.

It would be desirable if the detection of analytes in body perspiration could take place without the extensive use of machines and at the earliest possible stage of their appearance in body perspiration.

The published applications described in the following describe the coloring of fibers and pieces of fiber using pH indicator dyes by soaking the fibers with dye solutions and thus achieving physical adhesion of the dyes to the fibers or pieces of fiber. The execution of these technologies is games- or fashion-oriented.

In DE 1478608, with respect to a toy wig, the coloring of a hair-like fiber covering is described, wherein the “hair-like fiber covering” is colored using a dye which undergoes a color change when the pH value changes and thus enables a randomly repeatable color change by treating the fiber covering with liquids of different pH values.

DE 1603454 relates to DE 1478608 and extends the idea of the same by generally describing the coloring of fibers using pH indicator dyes.

Both published applications describe the coloring of fibers using pH indicator dyes, like, e.g., methyl red, alizarin or Congo red. The mentioned methods here acquire the coloring of complete fibers or also only the coloring of partial areas of the pieces of fiber, or the printing of patterns or samples. The presented fibers are acetate fibers, viscose fibers and cotton fabrics which are soaked with dye solutions, dried and cleaned according to the described methods. Due to the structure of the mentioned fibers, a physical adhesion of the pH indicator dyes to the fiber spacings is acquired, but the stability of the coloring is so low that in each washing process a removal of the colors from the fibers due to thinning effects will take place.

For diagnostic use, the mentioned technologies described are not suitable.

Apart from that, in connection with the normal optical coloring of textiles the use of permanent chemical bonding of dyes to fibers is known in order to acquire a permanent and stable color impression.

In DE 200 2005 006 986 U1 with the title “Dauerhaft gefärbte Textilien” (Permanently colored textiles) for example the chemical reaction of dyes with cotton fibers is described. The aim here is to acquire an especially high color fastness when coloring textiles.

It would thus be desirable, if possible, to be able to use body perspiration as a potential indicator of existing diseases in a person as early as possible without causing effort for the person and additionally use the same in everyday life.

### SUMMARY

An embodiment may have a piece of clothing to which an indicator dye is bonded by chemical bonding, wherein the piece of clothing comprises at least one fiber to which the indicator dye is indirectly coupled via nano- or microparticles which are bonded, printed, adhered or laminated to the at least one fiber, wherein the indicator dye is covalently bonded to the surface of the nano- or microparticles, is covalently bonded in pores of the nano- or microparticles, or the indicator dye is covalently polymerized into the nano- or microparticles.

The inventive piece of clothing includes an indicator dye which is bonded to a piece of clothing by means of chemical bonding.

The present invention is based on the finding that the easiest way for the corresponding person to use the indicating character of body perspiration with respect to possible diseases is to use a piece of clothing as a carrier for the corresponding indicator dye, i.e. without additional strain or stresses in everyday life or additional general effort, by the indicator dye being bonded to the piece of clothing by a chemical bond, such as an atomic bond or covalent bond. The chemical bond may be directly between the indicator dye and at least one fiber of the piece of clothing or the chemical bond may be indirect between the indicator dye and the piece of clothing, e.g. between the indicator dye and nano- or microparticles, which, in turn, may again be bonded in any way to the piece of clothing, like, e.g., chemically, mechanically or via an adhesive.

### BRIEF DESCRIPTION OF THE DRAWINGS

Embodiments of the present invention will be detailed subsequently referring to the appended drawings, in which:

FIG. 1 is a schematical diagram of a piece of clothing according to one embodiment; and

FIG. 2 is a schematical sectional view of a piece of clothing according to one embodiment.

### DETAILED DESCRIPTION OF THE INVENTION

In the following, with reference to the drawings, embodiments of pieces of clothing are described wherein indicator dyes are bonded to fibers or pieces of fiber by a chemical bond, like, e.g., with the aim of a diagnostic evaluation of the change in the optical characteristics of the indicator dyes in analyte detection or contacting a corresponding analyte.

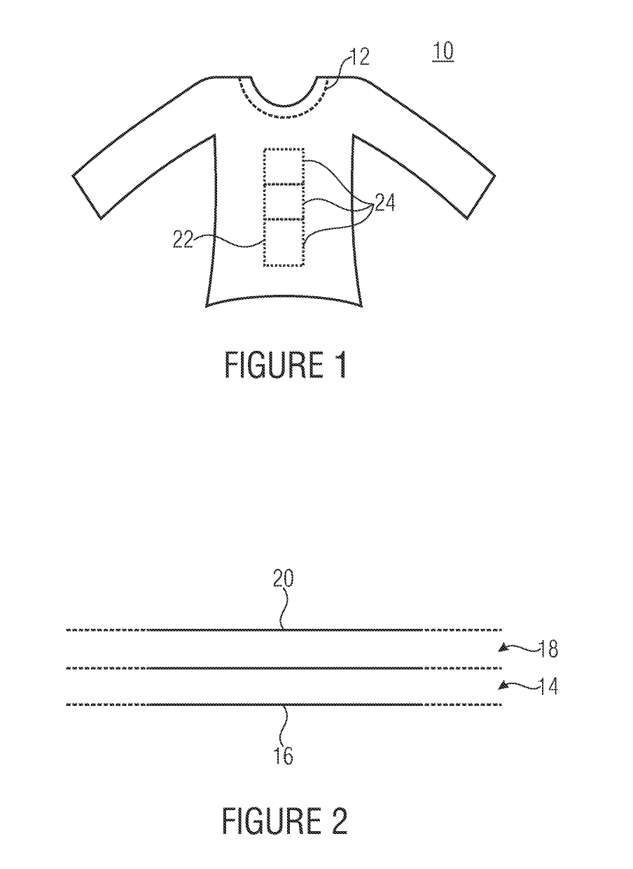

FIG. 1 shows, for example, a piece of clothing 10. In FIG. 1, the piece of clothing 10 is exemplarily illustrated as a top, but it could likewise be trousers, underpants, a vest, a T-shirt, a headband, a sweatband for the wrist, swimwear or the like.

The piece of clothing 10 includes at least one fiber to which an indicator dye is bonded by means of chemical bonding. For example, the piece of clothing 10 consists by less than 90% by weight of fibers to which the indicator dye is bonded. The piece of clothing 10 may, for example, consist exclusively of such fibers including or excluding the threads at optional seams 12 of the piece of clothing 10.

It may also, however, be the other way round, i.e. that the piece of clothing 10 comprises fibers provided with the indicator dye only at a seam or seams 12. The piece of clothing 10 may consist of several layers, e.g. comprise an inner lining and an outer lining, as is indicated in FIG. 2, where reference numeral 14 indicates a fabric which is adjacent to an interior side 16 of the piece of clothing 10, and reference numeral 18 indicates a fabric which is adjacent to the exterior side 20 of the piece of clothing. It may be the case that the fibers of the textile fabric 16 are provided with the indicator dye, so that when the piece of clothing 10 is worn by the person wearing the same, the indicator dye is in contact with the skin surface of the person so that the sensitivity of detection of the analyte in the body perspiration for which the indicator dye is sensitive is increased. The color change or other changes in the optical characteristics of the indicator dye when contacting the analyte is not visible from the outside to third parties in this case, which may be desirable for aesthetic or also data protection reasons—when used in supervised institutions. However, it may also be the other way round, i.e. that the textile fabric 16 is not provided with the indicator dye but implemented such that it carries body perspiration by capillary forces or other physical characteristics with respect to the fabric 20, which is again provided with the indicator dye, so that its color change or other changes in optical characteristics are visible to third parties from the outside when the analyte is contacted in body perspiration. Although sensitivity with respect to the analyte would all in all be slightly reduced due to the indirect contact of the skin surface, the advantage would be that the indicator dye of the textile fabric 20 would not directly contact the skin in this case and would thus possibly be subjected to less wear, whereby, due to the wear, there would be a risk of the indicator dye contacting the skin surface of the person. As already mentioned above, it would also be possible for a textile fabric provided with the indicator dye to be continuously present from the interior to the exterior side 20, i.e. that the piece of clothing 10 more or less completely consists of this fabric.

It is further possible, however, that only parts of the piece of clothing are provided with a fiber textile fabric, i.e. only certain lateral sections, such as the sleeve, a certain back part or the like, which may be sewn up with the rest of the piece of clothing 10 or be otherwise connected.

In the latter way it would also be possible for different locations or sections of the piece of clothing to be provided with indicator dyes which are selectively sensitive with respect to different analysts, wherein examples of different analytes and indicator dyes are provided in the following. By the local separation of these different indicator dyes it would be possible to prevent an overlaying of the different optical changes in characteristics of the different indicator dyes from the point of view of the observer. Examining the changes in optical characteristics may rather be distributed to different sections of the piece of clothing 10, like, e.g., to different sections which are arranged along the back part in a spinal direction one after the other. The sections may be separated by markings optically visible from the outside by an imprint or corresponding seams or the like, wherein such delimitation markings are exemplarily indicated in FIG. 1 by 22 and the individual sections are each indicated by a respective indicator dye by reference numeral 24.

With a piece of clothing according to one of the preceding embodiments, it is possible to more easily utilize a diagnostic exploitation of the informative value of a person's body perspiration. Easy applicability is supported by a stable chemical bonding of the indicator dye to the fibers. The fibers not only experience physical adherence but a chemical bonding to the fibers. The fibers or fiber pieces are washable for example at 60° to 90° without the color bleaching. By the chemical bonding of the reactive indicator dyes to the fibers, the fibers are permanently colored with the respective indicator dye. Embodiments of the present invention are thus suitable for diagnostic purposes. The suitability for diagnostic purposes is facilitated by the fact that the fibers or fiber pieces are chemically bonded to indicator dyes as washing out of the indicator dyes is not possible.

As mentioned above, indicator dyes can be used which selectively react with different analytes. A pH indicator dye may, for example, be used as an indicator dye, i.e. an indicator dye which is sensitive with respect to free H⁺ ions or free OH⁻ ions. The use of such a pH indicator dye for example enables giving information on water loss of the person wearing the piece of clothing, who may, for example, be an active person, such as an athlete. The visible color change may here, as mentioned above, be detected or assessed by the user himself or herself or by other persons without having to use instruments. The use of other indicator dyes for detecting analytes, like, e.g., sodium ions, glucose, lactate, fructose, reactive oxygen species, thiols, peptides, proteins, toxins, alcohols or amines, may contribute to an early detection of certain diseases.

The piece of clothing may, for example, be one which is worn regularly by a person at certain intervals, for example once a month over a certain period of time, like, e.g., pajamas on certain nights. On the basis of possibly occurring colorings which point to the existence of certain analytes in perspiration, early detection of diseases can thus be enabled and the person can contact a doctor about the further procedure.

The use of a piece of clothing 10 in hospital and care areas would also be advantageous. Patients or persons needing care can be monitored with respect to their health state by wearing such indicator textiles, continuously or intermittently. Also here, early detection of certain diseases or a change in the state of health would be possible.

Depending on the analytes to be detected, indicator dyes may be used which show changes in the optical characteristics on contact with the respective analytes. The analytes may, for example, be the pH value, saccharides, amines, alcohol, reactive oxygen species, thiols, peptides, proteins, toxins or ions.

The change in the optical characteristics may be detected, for example, by the change in the absorption wavelength, absorption intensity, emission wavelength, emission intensity or fluorescence decay time.

The chemical binding of the reactive indicator dyes may take place, e.g. by chemical reaction of the OH groups of the cellulose fibers with vinyl sulfonyl groups of the indicator dyes. For this purpose, e.g. 1.5-0.02·x milligrams of the reactive indicator dye mixed with x microliters of a concentrated sulfuric acid (with, for example, x=25) and left untouched for 10 to 60 min., e.g. 30 min., at ambient temperature. Then the mixture is put into y milliliters with, e.g., y>x/10, e.g. y=10, of distilled water and neutralized by adding z₁ microliters of z₂% sodium hydroxide solution (with, e.g., z₁·z₂≧100·y, wherein, e.g., z₁=100 and z₂=32). The cellulose (fiber) is added to this solution. After 2-20 min., e.g. 5 min., a solution of z₃ grams of sodium carbonate is added to z₄ milliliters of distilled water, whereby, e.g., with z₃>x/100 and z₄>x/16, e.g. z₄=10 and z₃=1.0). After further 2-20 min., e.g. 5 min., 5·z₁ microliters of a z₂% sodium hydroxide solution are added, wherein after 30 to 200 min., e.g. 60 min., the cellulose (fiber) is washed with distilled water. By this method, a covalent coloring of fibers, pieces of fiber and textiles with indicator dyes is acquired.

According to another method, the stable chemical bonding of indicator dyes to fibers may be acquired, e.g., by the use of amino-functionalized fibers and the chemical bonding of an acid function of the indicator dye to the amino groups of the fibers. The thus acquired amide bonding is very stable and also causes permanent coloring of the fibers, pieces of fiber and textiles with the indicator dyes.

One advantage of the above fibers, pieces of fiber or clothing textiles equipped with chemically bonded indicator dyes is the easy handling and the high information content, for example of the color change. A color change is easily detectable and information which is easy to interpret. It thus becomes possible for normal users, without having to use a lot of instruments and without detailed background knowledge, to supervise their own state of health or the state of health of the person needing care, for example in hospital or in the care area.

In view of the location of the use of the fibers, pieces of fiber and clothing textiles which are equipped with chemically bonded indicator dyes, the chemical bond offers an important advantage and thus very high stability of the coloring. The fibers, pieces of fiber and clothing textiles may be worn directly on the skin surface of the persons as, due to the stable adherence of the fibers, dyes are not worn off.

Further, the indicator dyes may operate reversibly. For example, they may be restored to their original state by washing. Washing may take place at temperatures up to 90° C. as the indicators are chemically bonded to the fibers. The indicator dyes may also operate irreversibly and thus cause permanent color change.

A further advantage is the possibility of the early detection of diseases by corresponding indicators being bonded to the fibers.

In contrast to previous implementations, it the case according to embodiments of the present invention that indicator dyes are not indirectly but directly bonded to the fibers by chemical bonding, such as by being bonded covalently to nano- and microparticles which are, for example, based on cellulose, dextran, polyacrylamide, silica, gold, silver or quantum dots or consist of the same, wherein these particles in turn are again bonded, printed, adhered or laminated to the fibers or textiles covalently or non-covalently or otherwise connected to the piece of clothing. The nano- and microparticles comprise, for example, an average diameter which is between 10 nanometers and 100 micrometers inclusively. The nano-/microparticles may be shaped spherically. To bond the dyes to nano- and microparticles in a chemically stable way, they may, for example, be bonded covalently to the surface or in pores of the particles. It is alternatively possible for nano- and microparticles to be covalently polymerized into the particles when manufacturing the particles. By this, the particles may be covered with the dye in a separate manufacturing process and subsequently be printed onto the textiles in a conventional textile printing method.

Fibers, fiber pieces and textiles according to the above embodiments may thus be utilized for the simple diagnostics of fitness and/or the state of health.

Above, covalent bonding was described as a form of chemical bonding which may serve to bond the indicator dye to the fiber. This bond may also be described as an atomic bond wherein an atom of the indicator dye and an atom of the fiber each contribute one electron—in the same or different proportions—to the bond, so that the electron pair belongs to both atoms.

While this invention has been described in terms of several embodiments, there are alterations, permutations, and equivalents which fall within the scope of this invention. It should also be noted that there are many alternative ways of implementing the methods and compositions of the present invention. It is therefore intended that the following appended claims be interpreted as including all such alterations, permutations and equivalents as fall within the true spirit and scope of the present invention.

1. A piece of clothing to which an indicator dye is bonded by chemical bonding, wherein the piece of clothing comprises at least one fiber to which the indicator dye is indirectly coupled via nano- or microparticles which are bonded, printed, adhered or laminated to the at least one fiber, wherein the indicator dye
is covalently bonded to the surface of the nano- or microparticles,
is covalently bonded in pores of the nano- or microparticles, or
the indicator dye is covalently polymerized into the nano- or microparticles.

2. The piece of clothing according to claim 1, wherein the indicator dye is implemented such that an optical characteristic of the indicator dye selectively changes when contacting an analyte.

3. The piece of clothing according to claim 2, wherein the indicator dye is implemented such that the optical characteristic that changes selectively on contact with the analyte, is an absorption wavelength, an absorption intensity, an emission wavelength, an emission intensity or a fluorescence decay time of the indicator dye.

4. The piece of clothing according to claim 1, wherein the indicator dye is implemented such that the change is reversible by washing out the analyte.

5. The piece of clothing according to claim 1, wherein the chemical bond is an atomic bond.

6. The piece of clothing according to claim 1, wherein the at least one fiber is exposed at an interior side (16) of the piece of clothing, so that when the piece of clothing is worn by a person the indicator dye is in contact with a skin surface of the person.

7. The piece of clothing according to claim 1, wherein the at least one fiber is exposed at an exterior side (20) of the piece of clothing, so that when the piece of clothing is worn by a person the change in the optical characteristic of the indicator dye is optically visible to a third person.

8. The piece of clothing according to claim 1, wherein the analyte is selected from a group comprising a pH value, saccharides, amines, sodium ions, glucose, lactate, reactive oxygen species, thiols, peptides, proteins, toxins, alcohols and fructose.

9. The piece of clothing according to claim 1, wherein the at least one fiber is a cellulose fiber and the chemical bond results from a chemical reaction of OH groups of the cellulose fiber with a vinylsulfone group of the indicator dye.

10. The piece of clothing according to claim 1, wherein the at least one fiber is an amino functionalized fiber and the chemical bond is an amide bond between an acid function of the indicator dye and an amino group of the amino functionalized fiber.
